(19) United States
(12) Patent Application Publication
(10) Pub. No.: US 2017/0266038 A1
(43) Pub. Date: Sep. 21, 2017

(54) UPPER BODY MOUNTED COOLING DEVICE

(71) Applicants: Clyoni Peavy, Gainesville, FL (US); Keoni Peavy, Gainesville, FL (US)

(72) Inventors: Clyoni Peavy, Gainesville, FL (US); Keoni Peavy, Gainesville, FL (US)

(21) Appl. No.: 15/610,672

(22) Filed: Jun. 1, 2017

Related U.S. Application Data
(63) Continuation-in-part of application No. 14/545,094, filed on Mar. 26, 2015.

(51) Int. Cl. A61F 7/00

(52) U.S. Cl. CPC A61F 7/0085; A61F 2007/0065; A61F 2007/0011

(57) ABSTRACT

A personal cooling device includes a rigid ventilation tube that is curved in part to fit around the neck of a human user such that the entire device may be supported solely in this manner. A powered fan is located at one end of the ventilation tube and configured to force air through the ventilation tube. Ventilation holes are arranged to direct air leaving the ventilation tube toward a user's body. A hollow compartment is provided in the ventilation tube adjacent the fan and is configured to received a portion of ice or other frozen material as a frozen liquid-filled sponge to effect evaporative cooling of passing air.

### REFERENCE TO PRIOR APPLICATIONS

This application is a Continuation-in-part application claiming benefit of pending U.S. application Ser. No. 14/545,094 filed on Mar. 26, 2015 by the applicants herein.

### BACKGROUND

The present invention relates generally to a wearable personal upper body mounted cooling device for persons suffering from hot flashes due to menopause or similar conditions. Individuals who suffer from hot flashes due to menopause, often wish to be cool especially in the summer time, the desire to remain cool may arise from environmental conditions outdoors or indoors, while walking or just simply attending a sporting event. Traditional cooling devices, that intake warm air and blow directly on the user's body, are generally heavy and bulky when worn by the users. As a result these prior devices are often worn only for a short time period. What is needed is a personal body cooling device that is easily and comfortably worn on a user's body.

### SUMMARY OF THE INVENTION

The present invention includes a rigid ventilation tube that is curved in part to fit around the neck of a human user such that the entire device may be supported solely in this manner. A powered fan is located at one end of the ventilation tube and configured to force air through the ventilation tube. Ventilation holes are arranged to direct air leaving the ventilation tube toward the user's body. A hollow compartment is provided in the ventilation tube adjacent the fan and is configured to received a portion of ice or frozen liquid-filled sponge to effect evaporative cooling of passing air during use. Clamps or other fastening devices may be used to secure the device to a user's clothing.

In preferred embodiments, the device includes a rotatable arm portion of the ventilation tube. The arm portion allows the user to adjust the geometry of the ventilation tube to direct cooling air in different directions. Alternatively, the ventilation tube is formed of a flexible material or construction to allow the user to alter the shape of the ventilation tube.

### DESCRIPTION OF THE DRAWINGS

FIG. 1 depicts one embodiment of the inventive device.

FIG. 2 depicts one manner of use of the inventive device worn on a user.

FIG. 3 depicts the inventive device in a first adjusted configuration on a user.

FIG. 4 depicts the device in a second adjusted configuration.

### DETAILS OF EMBODIMENTS OF THE INVENTION

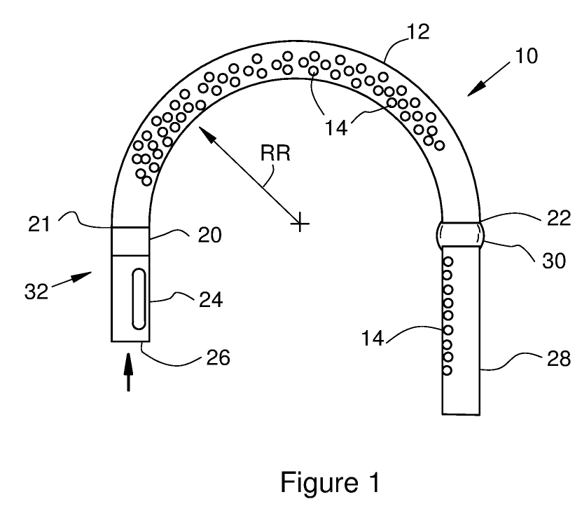

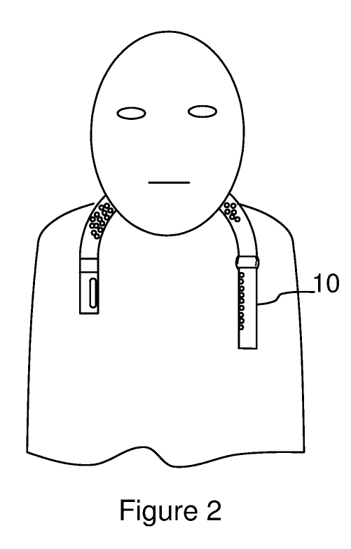

FIGS. 1 to 4 illustrate a common embodiment of the present invention and the following discussion regards all of the figures. The inventive device includes an elongated rigid ventilation tube 10 formed preferably of hollow tube plastic such as PVC or ABS plastic or similar materials typically used to fabricate such consumer articles. Most preferably, the ventilation tube 10 has a circular cross-section shape with a corresponding cross-section diameter in the range of one-half to two inches. The cross-section size being limited only by the need to convey sufficient air volume at a reasonable velocity for cooling function and comfort. While other cross-section shapes are possible, a circular shape is preferred for maximum esthetics and comfort. In alternative configurations, the ventilation tube 10 is formed of flexible material allowing the user to alter the shape of the ventilation tube from any of a variety of forms such as straight, curved, or circular.

The ventilation tube 10 includes a curved neck portion 12. The neck portion 12 is formed in a circular or near circular arc along its length. The curvature is configured to allow, during use of the device, the neck portion 12 to loosely encircle the back of the neck of an average human user. For this purpose, the neck portion 12 should extend between its two ends 20, 21 in a curve having a radius of curvature RR in the range of 3 to 4 inches. Larger configurations may be used to accommodate persons with abnormally large necks, but the specified dimension is applicable for a practical design serving most people. A substantially larger curve will result in a device that is unstable on a user and will not be easily maintained in the desired position.

Extending from the second end 22 of the neck portion 12 is an arm portion 28 of the ventilation tube 10. The length of the arm portion 28 may vary depending on the desires of the user. Both the neck portion 12 and the arm portion 28 include multiple air distribution apertures or holes 14 that are sized and relatively located to allow air forced through the ventilation tube 10 to exit in a direction to impinge on the user on whom the device is worn as described. Specifically, the holes 14 should be located facing generally inward toward the center of curvature of the neck portion 12, with some variation for distribution. Otherwise, the particular geometry and location of the holes 14 is not fixed and may vary between embodiments.

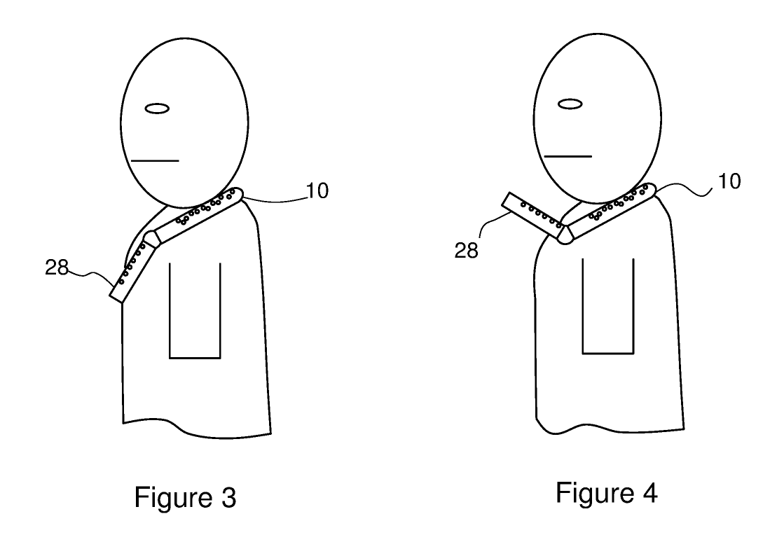

The arm portion 28 is preferably connected to the neck portion 12 through the use of a interconnecting rotatable joint 30 that allows the arm portion 28 to rotation out of the longitudinal axis of the neck portion at the second end 22 as illustrated in FIG. 3 and FIG. 4. Any of various different joint constructions and devices may be used to provide this rotation function and feature and the particular design is not critical.

A fan portion 32 of the device extends from the first end 21 of the ventilation tube 10. Preferably, the fan portion 32 has a cross-section geometry matching that of the neck portion 12. The fan portion 32 includes a hollow housing in which is mounted an electrically powered fan 20; the housing also forming a hollow coolant compartment 24. The terminal end 26 of the fan portion 32 is open to allow entry of surrounding ambient air into the coolant compartment 24. The coolant compartment 24 preferably includes an openable door configured to allow entry of a coolant article into the coolant compartment 24.

The fan portion 32 preferably includes an electrical power source such as batteries for powering the fan 20. The fan portion may include electrical controls such as power control switches. The fan 20 is preferably of bladed axial-flow design, but other types may be used.

In operation, the fan 20 is powered to draw air into the fan portion 32 through the terminal end 26. The air travels through the coolant compartment 24 and then through the fan 20. The air then flows into the neck portion 12 and a portion continues to the arm portion 28. The air escapes through the holes 14 to impinge on the user for cooling effect.

Preferably, during use, the cooling compartment 24 is provided by the user with an appropriate cooling article (not shown). The passing air is thereby cooled via evaporative cooling to increase cooling effect during use. The cooling article may take the form of an ice pack or frozen water-filled sponge or the like.

The above description is that of particular embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to elements in the singular, for example, using the articles “a,” “an,” “the” or “said,” is not to be construed as limiting the element to the singular.

We claim:

1. A personal cooling device comprising:
an elongated hollow tube configured to partially encircle a human neck, the tube having a first end and a second end and having multiple air distribution holes;
a powered fan connected to the first end and configured to force air into the tube to induce air to flow out of the distribution holes.

2. A personal cooling device, according to claim 1, and further comprising:
a fan portion connected to the first end and including the powered fan, the fan portion also including a hollow compartment.

3. A personal cooling device, according to claim 1, and wherein:
the tube further comprises an arm portion extending from the second end, the arm portion rotatable about a pivot point at the second end.

4. A personal cooling device, according to claim 2, and wherein:
the tube has a neck portion curving between the first and second ends and having a radius of curvature in the range of 3 to 4 inches.
